(19) United States
(12) Patent Application Publication
(10) Pub. No.: US 2005/0019397 A1
(43) Pub. Date: Jan. 27, 2005

(54) Cellulosic fiber containing composition

(76) Inventor: Richard L. Jacobs, Portage, MI (US)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 10/896,524

(22) Filed: Jul. 22, 2004

Related U.S. Application Data
(60) Provisional application No. 60/489,340, filed on Jul. 23, 2003.

(51) Int. Cl. A61K 9/48; A61K 9/20; A61K 9/14

(52) U.S. Cl. 424/464

(57) ABSTRACT

A process and pharmaceutical composition containing a dry granulated, preferably roller compacted, combination of cellulosic ether fiber and a binder is provided. The dry granulated combination of this invention may be combined with conventional disintegrants and other optional excipients, and compressed to provide rapidly disintegrating tablets.

### CROSS-REFERENCE TO RELATED APPLICATIONS

This application claims the benefit of U.S. Provisional Patent Application No. 60/489,340, filed Jul. 23, 2003, the disclosure of which is hereby incorporated by reference in its entirety.

### FIELD OF THE INVENTION

This invention relates to dietary cellulosic ether fiber compositions.

### BACKGROUND OF THE INVENTION

It is desirable to provide dietary cellulosic ether fiber compositions such as methycellulose, in tablet form. However, it is important that the tablets disintegrate readily in the digestive tract in order to make the cellulosic ether fiber available. The problem and difficulty of disintegrating methylcellulose based tablets was recognized in U.S. Pat. No. 4,017,598 to Ohno et al. as far back as Apr. 27, 1974. The Ohno et al. patent discloses solving the problem by wet granulating the methylcellulose and then combining those granules with a disintegrant. The process of wet granulation involves the steps of weighing and blending ingredients, mixing them in the presence of 9-11% water to form larger particles.

U.S. Pat. No. 6,350,469 to Daggy et al. acknowledges the common belief that tableted cellulose ethers do not readily dissolve in the digestive tract because they are highly hygroscopic, with the outer surface of the tablet forming a gel-like hydrate that prevents the tablet from breaking up and greatly retards the hydration of the inner portion of the tablet. Like Ohno '598, Daggy et al. addresses this problem by wet granulating the methylcellulose and combining the granules with a disintegrant. The examples exhibit a disintegration time of less than 10 minutes, and use a wet granulated methylcellulose with either a substantial amount of the disintegrant microcrystalline cellulose, or the combination of the disintegrant sodium starch glycolate with dicalcium phosphate.

### SUMMARY OF THE INVENTION

Contrary to convention, the inventors have found it unnecessary to wet granulate cellulosic ether fiber in order to obtain a rapidly disintegrating cellulosic ether fiber tablet. A cellulosic fiber containing composition in accordance with the present invention includes a dry granulated, preferably roller compacted combination comprising a cellulosic ether fiber and a binder.

These and other features, advantages, and objects of the present invention will be further understood and appreciated by those skilled in the art by reference to the following specification and claims.

### DETAILED DESCRIPTION OF PREFERRED EMBODIMENT

A preferred embodiment product of this invention, is a dry granulated combination including a pharmaceutically effective amount of a dietary cellulosic ether fiber, a binder and a very small amount of water. Other ingredients in the mix to be dry granulated are optional.

One process for dry granulation involves compacting a large mass of powdered, dry blended solids, and subsequently crushing and sizing the compacted mixture into smaller granules. Another, and more preferred process for dry granulation is roller compaction. With roller compaction, these powdered, dry blended solids are subjected to compaction mills to increase the density of the powder by passing it between high pressure rollers. This densified material is then broken up, sized and tablets are prepared by compression. It has been discovered that roller compacting dietary cellulosic ether results in the dietary cellulosic ether having a more uniform particle size and having better flow characteristics than similarly prepared wet granulated dietary cellulosic ether.

The roller compaction process is aided by the addition of 0.5 to 2.0% water. Roller compaction involves mixing the ingredients, and then roller compressing them into larger particles. In contrast to wet granulation, there is insufficient water present in the mixing step to significantly increase the particle size. This process may be achieved by using a Chilsonator® powder compressing machine made by Fitzpatrick Company, Elmhurst, Ill.

A preferred dietary cellulosic ether is methylcellulose. Methylcellulose may be administered to adult humans in a dosage regimen of 4 to 6 grams daily and to children (>6 years old) in a dosage regimen of 1-1.5 grams daily. The present invention may encompass many oral dosage forms including, but not limited to, caplets, tablets or any other compressed oral dosage forms. The amount of methylcellulose contained in each dosage form is preferably from about 100 mg to about 1000 mg, while the more preferred amount of methylcellulose per dosage form is about 250 mg to 750 mg. The compressed oral dosage form weight may be from about 200 mg to about 1500 mg, while the preferred compressed oral dosage form weight is from about 600 mg to about 800 mg.

The mix to be roller compacted includes a pharmaceutically effective amount of methylcellulose, a binder and a very slight amount of water. Preferably, the mix to be roller compacted may include a pharmaceutically effective amount of methylcellulose, an amount of maltodextrin that is effective to bind the methylcellulose, and an amount of water that is effective to aid compaction of the mix during roller compaction. Maltodextrin is both a binder and diluent. A suitable amount of maltodextrin that is effective to bind the methylcellulose is from about 5% to about 25% of the weight of the mix. A suitable amount of water that is effective to aid compaction during roller compaction is from about 0.5% to about 2% of the total weight of the mix, e.g., about 1%.

Any pharmaceutically acceptable grade of methylcellulose may be utilized in the present invention. Additionally, any pharmaceutically acceptable grade of any compressed oral dosage form excipients may be utilized in the present invention. Various pharmaceutical compressed oral dosage form excipients may be combined into the oral dosage form mixture, prior to compression, such as by using a V-shell blender or a rotating shell blender. Examples of such blenders include double-cone and twin shell blenders. Examples of types of excipients include, but are not limited to, fillers, binders, diluents, and processing aids, e.g. glidants, granulating agents, etc. Specific examples of excipients include, without limitation, maltodextrin (binder and/or diluent), povidone (binder), crospovidone (disintegrant), colloidal silica dioxide (surfactant and absorbant), dibasic calcium phosphate (filler), mannitol (filler), magnesium stearate (lubricant), sodium lauryl sulfate (surfactant), microcrystalline cellulose, lactose, starch, pregelatinized starch, sucrose, dextrose, corn syrup solids, stearic acid, colorants, etc.

The following examples illustrate a process for making rapidly disintegrating compositions containing methylcellulose. Disintegration and friability characteristics of an oral dosage form of the present invention are shown in Tables 1 through 5.

Tablet Preparation:

The process for making the invention includes making a mix to be roller compacted in an 1800 liter Fielder High Shear Granulator using the following materials:

| | |
|---|---|
| 3425 RM PURIFIED WATER | 3.50 KG |
| 25420 RM METHYLCELLULOSE USP 4000 | 297 KG |
| 3974 RM MALTODEXTRIN NF | 49.9 KG |

Because a small amount of water is added to the mix to be roller compacted, the Fielder High Shear Granulator is a suitable apparataus for preparing a mix to be roller compacted. Methylcellulose and maltodextrin are added to the Fielder granulator and mixed for 5 minutes. While the Fielder granulator continues to run, purified water is sprayed on to the powder and mixed for an additional 10 minutes. This continued mixing evenly distributes the water in the powders. Approximately 1% by weight of purified water is added to the methylcellulose and maltodextrin powders. The powders in the mix to be roller compacted have not increased significantly in particle size after mixing with water. The mix to be roller compacted is a very fine powder that has poor flow characteristics compared to the powder prior to mixing.

After mixing for 10 minutes the powder is discharged through a Comil grinder into poly-lined drums. The mix to be roller compacted is then roller compacted in a Chilsonator® powder compacting machine. As the compacted material comes out of the Chilsonator® powder compacting machine it is milled through a Fitzmill comminuting machine. The milled material is then sifted through a Sweco sifter. Two screens are used in the Sweco sifter with the top screen having larger holes and the bottom screen being a finer mesh. Particles too coarse to go through the top screen as well as the material going through the finer screen are recirculated with virgin material. The process continues until all of the virgin material has been processed. After roller compaction the powder is granular in appearance with good flow characteristics. At no time is the roller compacted material dried.

The roller compacted material is then mixed with the remaining ingredients to form the final mix. The remaining ingredients may include:

| Ingredient Name | % of Formula |
|---|---|
| colloidal silicone dioxide | 0.500 |
| crospovidone | 2.000 |
| mannitol | 12.264 |
| FD&C yellow #6 lake | 0.200 |
| magnesium stearate | 0.250 |
| sodium lauryl sulfate | 0.500 |

The roller compacted material is mixed with these remaining ingredients in a 75 cubic foot V-shell blender. After adequate blending has taken place, the final mix is compressed on a Mark IV Manesty tablet press to produce the desired oral dosage form.
Tablet Characteristics:

A rapidly disintegrating caplet of the invention will exhibit a low friability and a disintegration time of at least under 30 minutes. This is illustrated in Tables 1-5, which show the hardness, friability and disintegration time for 5 separate lots of tablets prepared in accordance with the examples.

TABLE 1

| | Weight | Thickness | Hardness |
|---|---|---|---|
| 1 | 0.725 | 0.223 | 15.5 |
| 2 | 0.719 | 0.223 | 14.1 |
| 3 | 0.726 | 0.223 | 15.9 |
| 4 | 0.728 | 0.223 | 15.4 |
| 5 | 0.728 | 0.223 | 14.6 |
| 6 | 0.730 | 0.223 | 15.1 |
| 7 | 0.721 | 0.222 | 14.8 |
| 8 | 0.719 | 0.223 | 15.0 |
| 9 | 0.727 | 0.223 | 16.2 |
| 10 | 0.714 | 0.222 | 14.0 |
| Mean | 0.724 | 0.223 | 15.1 |
| Std Dev | 0.005 | 0.000 | 0.718 |
| RSD | 0.714 | 0.189 | 4.770 |

| | |
|---|---|
| Friability | 0.00% |
| Disintegration | 27 minutes |

TABLE 2

| | Weight | Thickness | Hardness |
|---|---|---|---|
| 1 | 0.721 | 0.222 | 13.7 |
| 2 | 0.710 | 0.221 | 12.2 |
| 3 | 0.718 | 0.221 | 13.8 |
| 4 | 0.714 | 0.222 | 13.0 |
| 5 | 0.718 | 0.222 | 129 |
| 6 | 0.708 | 0.221 | 12.7 |
| 7 | 0.717 | 0.222 | 12.9 |
| 8 | 0.708 | 0.222 | 12.6 |
| 9 | 0.722 | 0.222 | 13.5 |
| 10 | 0.706 | 0.221 | 12.4 |
| Mean | 0.714 | 0.222 | 13.0 |
| Std Dev | 0.006 | 0.001 | 0.542 |
| RSD | 0.816 | 0.233 | 4.177 |

| | |
|---|---|
| Friability | 0.00% |
| Disintegration | 26 minutes |

TABLE 3

| | Weight | Thickness | Hardness |
|---|---|---|---|
| 1 | 0.720 | 0.233 | 10.1 |
| 2 | 0.708 | 0.232 | 9.0 |
| 3 | 0.715 | 0.232 | 10.6 |
| 4 | 0.720 | 0.232 | 10.1 |
| 5 | 0.709 | 0.232 | 9.3 |
| 6 | 0.716 | 0.232 | 9.8 |
| 7 | 0.719 | 0.232 | 10.0 |
| 8 | 0.715 | 0.232 | 9.8 |
| 9 | 0.711 | 0.231 | 9.6 |
| 10 | 0.717 | 0.232 | 10.0 |
| Mean | 0.715 | 0.232 | 9.8 |
| Std Dev | 0.004 | 0.000 | 0.450 |
| RSD | 0.611 | 0.203 | 4.576 |

| | |
|---|---|
| Friability | 0.11% |
| Disintegration | 20 minutes |

TABLE 4

| | Weight | Thickness | Hardness |
|---|---|---|---|
| 1 | 0.717 | 0.231 | 12.6 |
| 2 | 0.712 | 0.231 | 12.4 |
| 3 | 0.719 | 0.231 | 13.1 |
| 4 | 0.726 | 0.232 | 13.8 |
| 5 | 0.723 | 0.231 | 13.5 |
| 6 | 0.720 | 0.231 | 11.7 |
| 7 | 0.716 | 0.232 | 13.2 |
| 8 | 0.725 | 0.232 | 13.3 |
| 9 | 0.736 | 0.232 | 14.4 |
| 10 | 0.734 | 0.232 | 14.4 |
| Mean | 0.723 | 0.232 | 13.2 |
| Std Dev | 0.008 | 0.001 | 0.855 |
| RSD | 1.065 | 0.228 | 6.460 |

| | |
|---|---|
| Friability | 0.00% |
| Disintegration | 28 minutes |

TABLE 5

| | Weight | Thickness | Hardness |
|---|---|---|---|
| 1 | 0.712 | 0.233 | 7.7 |
| 2 | 0.708 | 0.233 | 7.4 |
| 3 | 0.712 | 0.233 | 7.5 |
| 4 | 0.704 | 0.233 | 7.7 |
| 5 | 0.710 | 0.232 | 7.5 |
| 6 | 0.712 | 0.233 | 7.4 |
| 7 | 0.691 | 0.233 | 6.7 |
| 8 | 0.702 | 0.233 | 6.6 |
| 9 | 0.704 | 0.233 | 7.4 |
| 10 | 0.698 | 0.233 | 6.9 |
| Mean | 0.705 | 0.233 | 7.3 |
| Std Dev | 0.007 | 0.000 | 0.399 |
| RSD | 0.987 | 0.136 | 5.487 |

| | |
|---|---|
| Friability | 0.00% |
| Disintegration | 18 minutes |

1. A composition comprising a dry granulated combination of a cellulosic ether fiber and a binder.

2. The composition of claim 1 wherein the dry granulated combination comprises a roller compacted combination.

3. The composition of claim 2 wherein said cellulosic ether fiber is methylcellulose.

4. The composition of claim 2 wherein the binder is maltodextrin.

5. The composition of claim 4 wherein the maltodextrin comprises from about 5% to about 25% of the weight of the dry granulated combination.

6. The composition of claim 2 wherein water comprises from about 0.5% to about 2.0% of the weight of the roller compacted combination.

7. The composition of claim 1 wherein the binder is maltodextrin.

8. The composition of claim 7 wherein the maltodextrin comprises from about 5% to about 25% of the weight of the dry granulated combination.

9. A solid compressed cellulosic ether fiber containing oral dosage form compressed from a composition comprising a dry granulated mixture of methylcellulose and a binder.

10. The solid compressed oral dosage form of claim 9 wherein said dry granulated mixture of methycellulose and a binder is roller compacted to form a roller compacted combination.

11. The solid compressed oral dosage form of claim 10 wherein said compressed oral dosage form also includes a disintegrant.

12. The solid compressed oral dosage form of claim 11 containing from about 100 mg to about 1000 mg of methylcellulose.

13. The solid compressed oral dosage form of claim 11 containing from about 250 mg to about 750 mg of methylcellulose.

14. The solid compressed oral dosage form of claim 11 further comprising at least one of povidone, crospovidone, mannitol, colloidal silicone dioxide, dibasic calcium phosphate, magnesium stearate, sodium lauryl sulfate, microcrystalline cellulose, lactose, starch, pregelatinized starch, sucrose, dextrose, corn syrup solids, stearic acid, or colorants.

15. The solid compressed oral dosage form of claim 11 further comprising a filler.

16. The solid compressed oral dosage form of claim 11 wherein the oral dosage form weight is from about 200 mg to about 1500 mg.

17. The solid compressed oral dosage form of claim 11 wherein the oral dosage form weight is from about 600 mg to about 800 mg.

18. The solid compressed oral dosage form of claim 10 wherein water comprises from about 0.5% to about 2.0% of the weight of the roller compacted combination.

19. The solid compressed oral dosage form of claim 18 wherein said compressed oral dosage form also includes a disintegrant.

20. The solid compressed oral dosage form of claim 19 further comprising a filler.

21. A process for preparing a cellulosic ether fiber containing composition comprising:
combining methylcellulose with a binder and from about 0.5 to about 2.0% water to provide a combination;
dry granulating the combination to form a dry granulated mix;
mixing with said dry granulated mix at least one disintegrant to form a final mix; and
compressing said final mix into a solid oral dosage form.

22. The process of claim 21 wherein dry granulating comprises roller compacting.

23. The process of claim 22 wherein the binder is maltodextrin.

24. The process of claim 23 wherein the maltodextrin comprises from about 5% to about 25% of the weight of the dry granulated mix.

25. The process of claim 21 wherein the final mix further comprises at least one of povidone, crospovidone, mannitol, colloidal silicone dioxide, dibasic calcium phosphate, magnesium stearate, sodium lauryl sulfate, microcrystalline cellulose, lactose, starch, pregelatinized starch, sucrose, dextrose, corn syrup solids, stearic acid, or colorants.
